(19) United States
(12) Patent Application Publication
(10) Pub. No.: US 2010/0234336 A1
(43) Pub. Date: Sep. 16, 2010

(54) Ophthalmic Compositions

(76) Inventors: Erning Xia, Penfield, NY (US); John Denick, JR., Jefferson City, TN (US); Richard V. Smerbeck, Pittsford, NY (US); Martin J. Coffey, Pittsford, NY (US)

Correspondence Address: Bausch & Lomb Incorporated, One Bausch & Lomb Place, Rochester, NY, 14604-2701, US

(21) Appl. No.: 12/732,639

(22) Filed: Mar. 26, 2010

Related U.S. Application Data
(63) Continuation-in-part of application No. 11/595,384, filed on Nov. 9, 2006.
(60) Provisional application No. 60/736,522, filed on Nov. 14, 2005.

(51) Int. Cl. A61K 31/566; A61P 27/02

(52) U.S. Cl. 514/179

(57) ABSTRACT

An aqueous ophthalmic composition comprises a carboxy-containing polyanionic polymer that has a viscosity in the range from about 300 cp to about 1500 cp outside the eye and a total concentration of cations of less than about 50 mM, wherein the viscosity of the composition does not increase when the composition comes into contact with surface ocular fluid.

### CROSS-REFERENCE

This application is a continuation-in-part patent application, and claims the benefit, of U.S. Provisional Patent Application No. 60/736,522 filed on Nov. 14, 2005 and U.S. patent application Ser. No. 11/595,384 filed on Nov. 9, 2006, the entire contents of which are incorporated by reference herein.

### BACKGROUND

The present invention relates to ophthalmic compositions and method of making and using the same. In particular, the present invention relates to ophthalmic compositions that are not readily eliminated from the eye and minimizes interference with vision. More particularly, the present invention relates to such ophthalmic compositions that further comprise at least a pharmaceutically active ingredient, and methods of making and using the same.

Ophthalmic compositions with or without pharmaceutically active ingredients (“APIs”) have been used to provide relief of a wide variety of conditions. Formulating ophthalmic compositions to achieve maximum beneficial effect without unavoidable disadvantages is a great challenge due to the unique physiological conditions of the eye. For example, many available ophthalmic compositions are solutions having low viscosity; e.g., less than about 30 cp (or mPa·s). When such a composition is applied to the eye, upon instillation, it is rapidly eliminated from the precorneal area of the eye because of lacrimal secretion and nasolacrimal drainage. As a result, it has been estimated that only 1-10% of the ophthalmic drugs can be utilized by patients, leading to necessary frequent instillation, or use of more concentrated solutions in order to achieve the beneficial effects.

To lengthen the retention time of instilled ophthalmic drug in the eye and to enhance the bioavailability of the ophthalmic drug, various ophthalmic vehicles have been developed. Examples of such ophthalmic vehicles include various inserts, ointments, suspension, and aqueous gels. However, these ophthalmic vehicles have their drawbacks. For example, the use of ointments often causes blurred vision. Also, insert is not particularly popular among patients due to its low patient compliance.

Among the ophthalmic vehicles, one kind, the so-called in situ gel-forming systems, has been particularly useful for prolonging precorneal retention time and improving ocular bioavailability of the ophthalmic drugs. Typically, in situ gel-forming systems are usually aqueous solutions and contain one or more polymers. These systems are low-viscosity liquid in the container and form gels on contact with tear fluid. The liquid-to-gel transition can be triggered by a change in temperature, pH, ionic strength, or the presence of tear proteins depending on the particular polymer system employed.

For example, A. Rozier et al., Int. J. Pharm. (1989), 57: 163-168, discloses a composition comprising an ion-activated gelling gellan gum (a polysaccharide) with the tradename of Gelrite® and an ion content below the gelation concentration. Rozier et al.'s gellan gum composition rapidly gels when mixed with simulated tear fluid having a combined concentration of mono- and divalent cations (sodium and calcium) of about 0.14 M.

U.S. Pat. No. 5,192,535 discloses an aqueous ophthalmic composition comprising a crosslinked carboxy-containing polymer. The composition has viscosity in the range of 1,000-30,000 cp and pH of 3-6.5, which rapidly gels (to viscosity of 75,000-500,000 cp) upon contact with the higher pH of tear fluid.

Joshi et al.'s U.S. Pat. No. 5,252,318 discloses reversibly gelling aqueous compositions which contain at least one pH-sensitive reversibly gelling polymer (such as carboxy vinyl linear or branched or cross-linked polymers of the monomers) and at least one temperature-sensitive reversibly gelling polymer (such as alkylcellulose, hydroxyalkyl cellulose, block copolymers of polyoxyethylene and polyoxypropylene, and tetrafunctional block polymers of polyoxyethylene and polyoxypropylene and ethylenediamine). It is contemplated that a high amount of salt (up to 0.2-0.9%) is used to have a low viscosity in the ungelled state. The compositions are formulated to have a pH of 2.5-6.5; preferably, 4-5.5. The viscosity of the compositions increases by several orders of magnitude (up to 1,000,000 cp) in response to substantially simultaneous changes in both temperature and pH.

U.S. Pat. No. 6,511,660 discloses a composition comprising Carbopol® and Pluronic® (a polyoxyethylene-polyoxypropylene copolymer) formulated at pH of 4. The composition turns into a stiff gel when in contact with physiological condition (37° C. and pH of 7.4).

Kumar et al., J. Ocular Pharmacol., Vol. 10, 47-56 (1994), discloses an ocular drug delivery system based on a combination of Carbopol and methylcellulose, prepared at pH of 4. This system turns into a stiff gel when the pH is increased to 7.4. Kumar et al., J. Pharm. Sci. Vol. 84, 344-348 (1995), discloses yet another ocular drug delivery system containing Carbopol® and hydroxyproplymethylcellulose, also prepared at pH of 4. This system turns into a stiff gel when the pH is increased to 7.4 and the temperature to 37° C. In both systems, a viscosity-enhancing polymer (methylcellulose or hydroxypropylmethylcellulose) is added in order to not have excessive amount of Carbopol® concentration without compromising the in situ gelling properties as well as overall rheological behaviors.

Finkenaur et al.'s U.S. Pat. No. 5,427,778 discloses a gel formulations contains a polypeptide growth factor and a water soluble, pharmaceutically or ophthalmically compatible polymeric material for providing viscosity within various ranges determined by the application of the gel. Both Carbopol® gels and Pluronic® gels, respectively, are disclosed in the patent. Pluronic® is the trademark for BASF's polyoxyethylene-polyoxypropylene block copolymers.

These exemplary and other prior-art compositions all have a common characteristic of having a low viscosity in the container and becoming a stiff gel upon being instilled in the eye due to an increase in at least one of pH, temperature, and ionic strength. Although a stiff gel can have an extended residence in the eye and assist in promoting a higher drug bioavailability, such gel can interfere adversely with vision. In addition, these prior-art compositions must often be formulated at significantly acidic pH, which is not comfortable upon installation in the eye of the patient.

Therefore, it is desirable to provide ophthalmic compositions that can mitigate or avoid these shortcomings.

### SUMMARY

In general, the present invention provides a topical ophthalmic composition that is topically administrable into an eye of a subject as a drop and is substantially incapable (or incapable) of substantially increasing its viscosity after instilled in the eye. In one embodiment, the viscosity of the composition does not increase upon contact with the tear fluid in the eye. In another embodiment, the composition has a viscosity from about 300 cp (mPa·s) to about 1500 cp outside the eye, wherein the viscosity is measured at conditions disclosed hereinbelow.

In another aspect, the viscosity of the composition does not increase in the eye as determined by a subject feeling of a majority of test subjects who receive such a composition.

In still another aspect, a composition of the present invention comprises a gel formulation comprising a water-insoluble pharmaceutical active ingredient which does not settle out of the formulation upon storage for at least 1 month. As used herein, the term “water-insoluble” means having a solubility in water of less than or equal to 0.1 mg/g of water, as measured at 25° C. and pH of 7-7.5.

In another aspect, the present invention provides topical ophthalmic aqueous compositions comprising anionic polymers such as carboxy-containing polymers (e.g., carboxyvinyl polymers, polyacrylic acid, polymethylmethacrylic acid, and their derivatives), hyaluronic acid, alginates, carboxy methyl cellulose; osmotic agents (such as propylene glycol, glycerin, sugars, mannitol, amino acid; chelating agent such as EDTA, phosphonic acids, and salts thereof); and one or more water-insoluble APIs.

In yet another aspect, a topical ophthalmic composition of the present invention comprises a total concentration of cations of less than about 50 mM (or alternatively, less than about 40 mM, or less than about 30 mM, or less than about 20 mM, or less than about 10 mM).

Further disclosed are methods for producing such a topical ophthalmic composition. The method comprises incorporating or mixing a water-insoluble API in a pharmaceutically acceptable vehicle or carrier to produce a gel suspension, which vehicle comprises water and a polyanionic polymer such as carboxy-containing polymer or polyacrylic acid), which polyanionic polymer is present in the vehicle or carrier in an amount that provides the composition in the form of a gel, wherein the composition has a concentration of cations less than about 50 mM, and wherein the composition has a viscosity in the range from 300 cp to less than 1500 cp (or mPa·s) outside an eye of a subject. In one embodiment, such API comprises loteprednol etabonate or a pharmaceutically acceptable ester thereof.

### BRIEF DESCRIPTION OF THE DRAWINGS

FIG. 1 shows the Brookfield viscosity of a loteprednol etabonate 0.5% ophthalmic gel of the present invention.

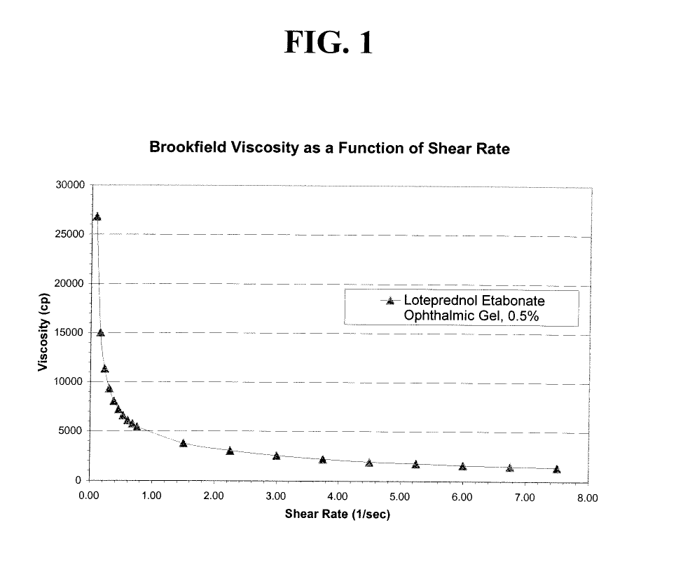

### DETAILED DESCRIPTION

In general, the present invention provides a topical ophthalmic composition that is topically administrable into an eye of a subject as a drop and is substantially incapable (or incapable) of substantially increasing its viscosity after instilled in the eye. In one embodiment, the viscosity of the composition does not increase upon contact with the tear fluid in the eye.

In one aspect, the composition has a viscosity in the range from about 300 cp to about 1500 cp outside the eye.

In another aspect, the viscosity of the composition does not increase in the eye as determined by a subject feeling of a majority of test subjects who receive such a composition.

Thus, the present invention is novel over the prior-art sustained release topical ophthalmic compositions in that a composition of the present invention at rest is a gel outside the eye and does not increase in viscosity upon contact with surface ocular fluid, whereas said prior-art compositions are typically low-viscosity solutions that gel (sometimes to stiff gels that can interfere with vision) upon contact with the ocular fluid.

In one aspect, a composition of the present invention can undergo shear-thinning in the eye.

As applied herein to the present invention, viscosity is measured with a Brookfield Engineering Laboratories RVDV-III Ultra C rheometer (a cone-and-plate rheometer) with CPE-40 spindle, at 25° C., and shear rate of 7±1 sec⁻¹.

In another aspect, the present invention provides topical ophthalmic aqueous compositions comprising anionic polymers such as carboxy-containing polymers (e.g., carboxyvinyl polymers, polyacrylic acid, polymethylmethacrylic acid, and their derivatives), hyaluronic acid, alginates, carboxy methyl cellulose; osmotic agents (such as propylene glycol, glycerin, sugars, mannitol, amino acid; chelating agent such as EDTA, phosphonic acids, and salts thereof); and one or more water-insoluble APIs.

In yet another aspect, a topical ophthalmic composition of the present invention comprises a total concentration of cations of less than about 50 mM (or alternatively, less than about 40 mM, or less than about 30 mM, or less than about 20 mM, or less than about 10 mM).

In a further aspect, a composition of the present invention comprises one or more water-insoluble steroids (in particular, safe steroids, as known by persons skilled in the art). In one embodiment, such steroid comprises loteprednol etabonate or a pharmaceutically salt or ester thereof.

Steroids for treating ocular inflammations can be based on predictably metabolized drugs. Predictably metabolized drugs, as is known in the art, are designed to provide maximal therapeutic effect and minimal side effects. By one approach, synthesis of a “predictably metabolized drug” can be achieved by structurally modifying a known inactive metabolite of a known active drug to produce an active metabolite that undergoes a predictable one-step transformation in-vivo back to the parent, inactive metabolite (see; e.g., U.S. Pat. Nos. 6,610,675, 4,996,335 and 4,710,495 for predictably metabolized steroids). “Predictably metabolized drugs” therefore are biologically active chemical components characterized by predictable in-vivo metabolism to non-toxic derivatives after they provide their therapeutic effect. Formulations of steroids suitable for ophthalmic use are known. For example, U.S. Pat. Nos. 4,710,495, 4,996,335, 5,540,930, 5,747,061, 5,916,550, 6,368,616 and 6,610,675, the contents of each of which is incorporated by reference herein, describe predictably metabolized steroids and/or formulations containing predictably metabolized steroids.

(11β,17α),-17-((Ethoxycarbonyl)oxy)-11-hydroxy-3-oxoandrosta-1,4-diene-17-carboxylic acid chloromethyl ester (loteprednol etabonate) is a known compound and can be synthesized by methods disclosed in U.S. Pat. No. 4,996,335, the entire contents of which are hereby incorporated by reference in the present specification.

According to the methods of the present invention, a formulation comprising (11β,7α),-17-((ethoxycarbonyl)oxy)-11-hydroxy-3-oxoandrosta-1,4-diene-17-carboxylic acid chloromethyl ester and a pharmaceutically acceptable carrier for topical ophthalmic administration or implantation into the conjunctival sac or anterior chamber of the eye is administered to a mammal in need thereof. The formulations are formulated in accordance with methods known in the art for the particular route of administration desired.

The formulations administered according to the present invention comprise a pharmaceutically effective amount of (11β,17α),-17-((ethoxycarbonyl)oxy)-11-hydroxy-3-oxoandrosta-1,4-diene-17-carboxylic acid chloromethyl ester. As used herein, a “pharmaceutically effective amount” is one which is sufficient to reduce or eliminate signs or symptoms of dry eye. Generally, for formulations intended to be administered topically to the eye in the form of eye drops or eye ointments, the amount of (11β,17α),-17-((ethoxycarbonyl)oxy)-11-hydroxy-3-oxoandrosta-1,4-diene-17-carboxylic acid chloromethyl ester will be about 0.001 to 5% (w/w). For preferred topically administrable ophthalmic formulations, the amount of (11β,17α),-17-((ethoxycarbonyl)oxy)-11-hydroxy-3-oxoandrosta-1,4-diene-17-carboxylic acid chloromethyl ester will be about 0.001 to 1% (w/w).

The formulations administered according to the present invention may also include various other ingredients, including but not limited to surfactants, tonicity agents, buffers, preservatives, co-solvents and viscosity-building agents.

Surfactants that can be used are surface-active agents that are acceptable for ophthalmic or otolaryngological uses. Useful surface active agents include but are not limited to polysorbate 80, tyloxapol, Tween® 80 (ICI America Inc., Wilmington, Del.), Pluronic® F-68 (from BASF, Ludwigshafen, Germany) and the poloxamer surfactants can also be used. These surfactants are nonionic alkaline oxide condensates of an organic compound which contains hydroxyl groups. The concentration in which the surface active agent may be used is only limited by neutralization of the bactericidal effects on the accompanying preservatives (if present), or by concentrations which may cause irritation.

Various tonicity agents may be employed to adjust the tonicity of the formulation. For example, sodium chloride, potassium chloride, magnesium chloride, calcium chloride, nonionic diols, preferably glycerol, dextrose and/or mannitol may be added to the formulation to approximate physiological tonicity. Such an amount of tonicity agent will vary, depending on the particular agent to be added. In general, however, the formulations will have a tonicity agent in an amount sufficient to cause the final formulation to have an ophthalmically acceptable osmolality (generally about 150-450 mOsm/kg). A nonionic tonicity agent is preferred. However, if an ionic compound is used to assist in adjusting the tonicity, such compound is used in an amount such that the total concentration of cations in a composition of the present invention is within the range herein disclosed.

An appropriate buffer system (e.g., sodium phosphate, sodium acetate, sodium citrate, sodium borate or boric acid) may be added to the formulations to prevent pH drift under storage conditions. The particular concentration will vary, depending on the agent employed.

Topical ophthalmic products are typically packaged in multidose form. Preservatives are thus required to prevent microbial contamination during use. Suitable preservatives include: biguanides, hydrogen peroxide, hydrogen peroxide producers, benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1% (w/w). Unit dose formulations of the present invention will be sterile, but typically unpreserved. Such formulations, therefore, generally will not contain preservatives.

Co-solvents and viscosity-building agents may be added to the formulations to improve the characteristics of the formulations. Such materials can include nonionic water-soluble polymer. Other compounds designed to lubricate, “wet,’ approximate the consistency of endogenous tears, aid in natural tear build-up, or otherwise provide temporary relief of dry eye symptoms and conditions upon ocular administration the eye are known in the art. Such compounds may enhance the viscosity of the formulation, and include, but are not limited to: monomeric polyols, such as, glycerol, propylene glycol, ethylene glycol; polymeric polyols, such as, polyethylene glycol, hydroxypropylmethyl cellulose (“HPMC”), carboxy methylcellulose sodium, hydroxy propylcellulose (“HPC”), dextrans, such as, dextran 70; water soluble proteins, such as gelatin; and vinyl polymers, such as, polyvinyl alcohol, polyvinylpyrrolidone, povidone and carbomers, such as, carbomer 934P, carbomer 941, carbomer 940, carbomer 974P. Other compounds may also be added to the ophthalmic formulations of the present invention to increase the viscosity of the carrier. Examples of viscosity-enhancing agents include, but are not limited to: polysaccharides, such as hyaluronic acid and its salts, chondroitin sulfate and its salts, dextrans, various polymers of the cellulose family; vinyl polymers; and acrylic acid polymers. However, when a viscosity-adjusting agent is used, its amount is such that the viscosity of a composition of the present invention is in the range from about 300 cp to about 1500 cp (or mPa·s) outside the eye (e.g., in a bottle) (or alternatively, from about 300 cp to about 1400 cp, or from about 300 cp to about 1300 cp, or from about 300 cp to about 1200 cp, or from about 500 cp to about 1300 cp, or from about 500 cp to about 1200 cp, or from about 1000 cp to about 1200 cp, or from about 1000 cp to about 1500).

Formulations formulated for the treatment of dry eye-type diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous formulations which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of an API for the treatment or relief of such condition. An example of such an API may be (11β,17α),-17-((ethoxycarbonyl)oxy)-11-hydroxy-3-oxoandrosta-1,4-diene-17-carboxylic acid chloromethyl ester. Examples of artificial tears formulations useful as artificial tears carriers include, but are not limited to, commercial products, such as Moisture Eyes™ Lubricant Eye props/Artificial Tears, Moisture Eyes™, Liquid Gel lubricant eye drops, Moisture Eyes™, Preservative Free Lubricant Eye props/Artificial Tears and Moisture Eyes™, Liquid Gel Preservative Free Lubricant Eye props/Artificial Tears (Bausch & Lomb Incorporated, Rochester, N.Y.). Examples of phospholipid carrier formulations include those disclosed in U.S. Pat. No. 4,804,539 (Guo et al.), U.S. Pat. No. 4,883,658 (Holly), U.S. Pat. No. 4,914,088 (Glonek), U.S. Pat. No. 5,075,104 (Gressel et al.), U.S. Pat. No. 5,278,151 (Korb et al.), U.S. Pat. No. 5,294,607 (Glonek et al.), U.S. Pat. No. 5,371,108 (Korb et al.), U.S. Pat. No. 5,578,586 (Glonek et al.), the contents of each of which are incorporated by reference herein.

The preferred formulations of the present invention are intended for administration to a human patient suffering from ophthalmic diseases such as dry eye or symptoms of dry eye. Preferably, such formulations will be administered topically. In general, the doses used for the above described purposes will vary, but will be in an effective amount to eliminate or improve dry eye conditions. Generally, 1-2 drops of such formulations will be administered from once to many times per day. The formulation is intended to be provided as a package for the treatment of dry eye, the package would include the pharmaceutical formulation comprising loteprednol etabonate contained in a pharmaceutically acceptable container; a written package insert containing instructions for using the formulation for the treatment of dry eye; and outer packaging identifying the pharmaceutical formulation contained therein. In certain embodiments wherein the formulation is preservative free, the package would contain a pharmaceutically acceptable container suitable for single use by a user of the packaged formulation. In such embodiments it is envisioned that the outer packaging would contain at least one pharmaceutically acceptable container containing the loteprednol etabonate formulation. Preferably the outer packing would contain a multiplicity of single use containers, for example, enough single use containers to provide for a one-month supply of the formulation.

In yet another aspect, the present invention provides a method of producing a sterile loteprednol etabonate gel that comprises polyacrylic acid, wherein loteprednol etabonate is separately sterilized and incorporated into a polyacrylic acid gel, in a suitable amount, under aseptic conditions. Alternatively, the sterile loteprednol etabonate is suspended with a part of the solution, which may contain a sterile tonicity agent, used for the production of the polyacrylic acid gel, and this suspension is then homogenously mixed in with the separately sterilized polyacrylic acid gel.

It has been shown that a sterile loteprednol etabonate gel in a polyacrylic acid-containing base can be satisfactorily produced when certain method steps are followed in its production. According to one embodiment of the present invention, an aqueous polyacrylic acid suspension is made and then autoclaved under sterile conditions. This polyacrylic acid suspension is mixed with a sterile-filtrated solution of preserving agent, isotonicity agent, and chelating agent. After careful and thorough mixing of the starting materials, the addition of sterile-filtrated caustic soda solution initiates gel formation, and the gel is further subjected to agitation until it is homogenous. Meanwhile loteprednol etabonate or its pharmaceutically acceptable ester is sterilized. This can be accomplished by dissolving the active substance in a suitable amount of solvent, for example ethyl acetate, subjecting the solution to sterile filtration, and precipitating the active substance, for example, through the addition of sterile water with an anti-microbial agent under aseptic conditions. The microbially sterile loteprednol etabonate or its pharmaceutically acceptable ester is then triturated or ground to a powder with about three to ten times that amount of the gel base. The remaining amount of gel is then incorporated in the concentrate by thorough mixing. The finished gel preparation is then conventionally decanted or drawn off under sterile conditions. In an alternative variation of this method, the microbially sterile loteprednol etabonate or its pharmaceutically acceptable ester can be, to a large extent, suspended in a part of the aqueous solution of the tonicity agent. The polyacrylate gel can be made in a conventional manner with the remaining amount of isotonic agent and separately the isotonic suspension of the loteprednol etabonate can be homogenously mixed with the polyacrylate under sterile conditions.

This sterile gel is well acceptable to the patient, because its application does not have the disadvantage of known ointments and is not oily. Stability has been proven, so that the gel has a relatively long shelf life without any change in its physical properties. In particular, there is no settlement of loteprednol etabonate from the gel upon storage (25-40° C.) for at least 2 months. In addition, no crystal growth of the active ingredient is observed. Such a sterile gel preparation represents a significantly improved form of application in the opthalmological field. The present invention will be further explained and illustrated by the Example that follows.

The invention will now be further described by way of several examples that are intended to describe but not limit the scope of the invention as defined by the claims herein.

Representative eye drop formulations are provided in Examples 1-4 below.

### Example 1

TABLE 1

| Ingredient | Amount | |
|---|---|---|
| Phase I | | |
| Carbopol 934P NF | 0.25 | g |
| (Acrylic acid-based polymer) | | |
| Purified water | 99.75 | g |
| Phase II | | |
| Propylene glycol | 5.0 | g |
| EDTA | 0.1 | mg |
| Loteprednol etabonate | 50.0 | g |

Mix five parts of phase II with twenty parts of phase I for more than 15 minutes (and up to 10 hours) and adjust pH to 6.2-6A using 1N NaOH.

### Example 2

TABLE 2

| Ingredient | Amount | |
|---|---|---|
| Phase I | | |
| Carbopol 934P NF | 0.25 | g |
| (Acrylic acid-based polymer) | | |
| Purified water | 99.75 | g |
| Phase II | | |
| Propylene glycol | 3.0 | g |
| Tracetin | 7.0 | g |
| Loteprednol etabonate | 50.0 | g |
| EDTA | 0.1 | g |

Mix five parts (by weight) of phase II with twenty parts (by weight) of phase I for more than 15 minutes (and up to 10 hours) and adjust pH to 6.2-6.4 using 1N NaOH.

### Example 3

TABLE 3

| Ingredient | Amount | |
|---|---|---|
| Phase I | | |
| Carbopol 934P NF | 0.25 | g |
| (Acrylic acid-based polymer) | | |
| Purified water | 99.75 | g |
| Phase II | | |
| Propylene glycol | 7.0 | g |
| Glycerin | 3.0 | g |
| Loteprednol etabonate | 5.0 | g |
| EDTA | 0.1 | mg |
| Benzalkonium chloride (“BAK”) | 0.1-0.2 | mg |

Mix one part (by weight) of phase II with twenty parts (by weight) of phase I for more than 15 minutes (and up to 10 hours) and adjust pH to 6.2-6.4 using 1N NaOH.

### Example 4

TABLE 4

| | | | Amount Range | |
|---|---|---|---|---|
| | | | (per 100 g of total | |
| Ingredient | Amount | | composition) | |
| Phase I | | | | |
| Polycarbophil Noveon ® AA-1 | 0.375 | g | 0.2-0.5 | g |
| (Acrylic acid-based polymer) | | | | |
| Purified water | 99.625 | g | q.s. to 100 g of | |
| | | | combined composition | |
| | | | of 1 part (by weight) of | |
| | | | phase I and 1 part by | |
| | | | weight) of phase II | |
| Phase II | | | | |
| Propylene glycol | 0.44 | g | 0.3-0.6 | g |
| Glycerin | 0.88 | g | 0.6-1 | g |
| Loteprednol etabonate | 0.5 | g | 0.3-2 | g |
| Edetate disodium dihydrate | 0.055 | g | 0.03-0.07 | g |
| Tyloxapol | 0.05 | g | 0.03-1 | g |
| Boric acid | 0.5 | g | 0.3-0.6 | g |
| Benzalkonium chloride | 0.006 | g | 0.003-0.01 | g |
| (“BAK”) | | | | |

Mix one part (by weight) of phase II with one part (by weight) of phase I for more than 15 minutes (and up to 10 hours) and adjust pH to 6.3-6.6 using 2N NaOH (for the foregoing formulation, about 1.6-1.7 g of 2N NaOH is adequate). The formulation has an osmolality of about 285 mOsm/kg and a viscosity of 1140 cp, as measured by Brookfield rheometer at conditions as disclosed hereinabove.

### Example 5

This prophetic example illustrates a method of making a gel according to the present invention, although the production of larger amounts of gel may be necessary to meet commercial demands. In the present example, the gel is produced with water that is suitable for injection purposes (injection grade). To produce 500 g of polyacrylate gel, 1.22 g of polyacrylic acid (packaged under the trademark Noveon® AA-1 Polycarbophil) is carefully suspended, with the aid of an ultrasonic apparatus, in about 700 ml water and autoclaved for 20 minutes at 121° C. and 2 bar absolute pressure (about 202 kPa). In 700 ml of sterile injection-grade water is then dissolved 0.050 g of benzalkonium chloride (BAK), 20 g sorbitol and 0.05 g of sodium EDTA dihydrate, which is then subjected to sterile filtering (Sartorius® cellulose nitrate filter, order no. 11307-50ACN, 0.2 μm) into a sterile vessel. This sterile-filtered solution is then mixed, with strong agitation, into the autoclaved polyacrylic acid suspension. Sterile water in the amount of 1958.121 g is then added, and the solution is subjected to further agitation for 5 to 10 minute. Subsequently, strong sodium hydroxide in the amount of 0.465 g is dissolved in exactly 40 g of injection-grade water. This caustic soda is then introduced drop-wise under agitation over a sterile filter (Millex-GS, 0.22 μm, SLGS 025 BS der Fa Millipore). The mixture is agitated until the formation of a completely homogenous gel.

A microbially sterile loteprednol etabonate in the amount of 5 g (or a different amount is used for a different desired strength) is then slowly and carefully mixed with about 30 to 50 g of the gel. The gel is subjected to sterile filtration of the solution, and separation with water containing a bacteriocide under sterile conditions. After the Loteprednol etabonate is accordingly suspended in the given amount of gel, the rest of the gel, in total 495 g, is carefully incorporated into the initial material. All method steps are carried out under aseptic conditions.

The prepared gel is likewise drawn off in tubes under aseptic conditions. By an alternative method, the microbially sterile loteprednol etabonate is suspended in a sterile-filtrated isotonic solution of 700 ml water, 0.05 g benzalkonium chloride, 20 g sorbitol and 0.05 g of disodium EDTA. This solution is then, as already described, incorporated, under strong agitation, in the autoclaved polyacrylate suspension. Further adaptation or modification of the invention, corresponding to the described production of sterile polyacrylic acid gel, falling within the scope of the following claims may occur to the skilled artisan.

### Stability of Composition of the Present Invention

Compositions of the present invention exhibited excellent stability upon long-term storage. The active pharmaceutical ingredients remained suspended in the vehicles upon storage at 40° C. for 2 months.

A formulation (code name: 2593-YH-151-3) was prepared according to Example 4 that had a target concentration of loteprednol etabonate of 6 mg/g. Samples of the formulation were stored in vials at 25° C. (code name: 2593-YH-151-3@25°) and 40° C. (code name: 2593-YH-151-3@40°). The concentrations of loteprednol etabonate of two samples and of aliquots of the same drawn from the top, middle, and bottom of a vial were measured when they were first prepared. The concentrations of loteprednol etabonate were measured again 2 and 3 months after preparation for aliquots drawn from the top, middle, and bottom of a vial. The concentrations were determined in duplicates by liquid chromatography, as known in the art, and are presented in the following tables.

TABLE 5
Initial Loteprednol Etabonate Concentration Measurement

| | | | Loteprednol |
|---|---|---|---|
| | | | Etabonate |
| | Retention | Peak Area | Amount (% |
| | Time | (arbitrary | of target |
| Sample Name | (minutes) | units) | concentration) |
| 25993-YH-151-3@25° | 8.958 | 5450826 | 102.665 |
| 25993-YH-151-3@25° | 8.952 | 5455208 | 102.748 |
| 25993-YH-151-3@40° | 8.948 | 5487701 | 102.160 |
| 25993-YH-151-3@40° | 8.950 | 5477384 | 101.968 |
| 25993-YH-151-3@25°_Top | 8.950 | 5537072 | 102.586 |
| 25993-YH-151-3@25°_Top | 8.951 | 5543652 | 102.708 |
| 25993-YH-151-3@25°_Middle | 8.939 | 5422457 | 101.365 |
| 25993-YH-151-3@25°_Middle | 8.921 | 5434169 | 101.584 |
| 25993-YH-151-3@25°_Bottom | 8.932 | 5428713 | 102.464 |
| 25993-YH-151-3@25°_Bottom | 8.938 | 5411897 | 102.146 |
| 25993-YH-151-3@40°_Top | 8.944 | 5384362 | 100.780 |
| 25993-YH-151-3@40°_Top | 8.927 | 5398005 | 101.036 |
| 25993-YH-151-3@40°_Middle | 8.925 | 5352920 | 101.421 |
| 25993-YH-151-3@40°_Middle | 8.934 | 5378600 | 101.907 |
| 25993-YH-151-3@40°_Bottom | 8.925 | 5584349 | 102.094 |
| 25993-YH-151-3@40°_Bottom | 8.915 | 5582383 | 102.059 |

TABLE 6
Loteprednol Etabonate Concentration Measurements
After 2 and 3 Months Storage at 40° C.

| | | | |
|---|---|---|---|
| | | | Loteprednol |
| | | | Etabonate |
| | Retention | Peak Area | Amount (% |
| | Time | (arbitrary | of target |
| Sample Name | (minutes) | units) | concentration) |

After 2 months in storage

| | | | |
|---|---|---|---|
| 25993-YH-151-3@40°- | 8.946 | 5542070 | 103.959 |
| 2M_Top | | | |
| 25993-YH-151-3@40°- | 8.946 | 5525356 | 103.646 |
| 2M_Top | | | |
| 25993-YH-151-3@40°- | 8.954 | 5207352 | 100.750 |
| 2M_Middle | | | |
| 25993-YH-151-3@40°- | 8.932 | 5288222 | 102.315 |
| 2M_Middle | | | |
| 25993-YH-151-3@40°- | 8.933 | 5406181 | 103.536 |
| 2M_Bottom | | | |
| 25993-YH-151-3@40°- | 8.927 | 5337770 | 102.226 |
| 2M_Bottom | | | |
| Mean | | | 102.7 |
| Standard deviation | | | 1.2 |

After 3 months in storage

| | | | |
|---|---|---|---|
| 25993-YH-151-3@40°- | 8.781 | 5654618 | 105.035 |
| 3M_Top | | | |
| 25993-YH-151-3@40°- | 8.767 | 5651770 | 104.982 |
| 3M_Top | | | |
| 25993-YH-151-3@40°- | 8.761 | 5555883 | 102.133 |
| 3M_Middle | | | |
| 25993-YH-151-3@40°- | 8.768 | 5513799 | 102.352 |
| 3M_Middle | | | |
| 25993-YH-151-3@40°- | 8.781 | 5407950 | 102.956 |
| 3M_Bottom | | | |
| 25993-YH-151-3@40°- | 8.769 | 5417098 | 103.130 |
| 3M_Bottom | | | |
| Mean | | | 103.6 |
| Standard deviation | | | 1.1 |

The foregoing data show that compositions of the present invention exhibit both good chemical and physical stability upon long-term storage even at an aggressive temperature condition. The active ingredient loteprednol etabonate continued to be suspended uniformly throughout the container. In other words, there is no settling of loteprednol etabonate from the vehicle and there is no indication of any breakdown of the active ingredient.

This invention has been described by reference to certain preferred embodiments; however, it should be understood that it may be embodied in other specific forms or variations thereof without departing from its special or essential characteristics. The embodiments described above are, therefore, considered to be illustrative in all respects and not restrictive, the scope of the invention being indicated by the appended claims rather than by the foregoing description.

The claims, as originally presented and as they may be amended, encompass variations, alternatives, modifications, improvements, equivalents, and substantial equivalents of the embodiments and teachings disclosed herein, including those that are presently unforeseen or unappreciated.

What is claimed is:

1. An aqueous ophthalmic composition comprising carboxy-containing polyanionic polymer, wherein the composition has a viscosity in the range from about 300 cp to about 1500 cp, a total concentration of cations of less than about 50 mM, and the viscosity does not increase upon contact with surface ocular fluid.

2. The composition of claim 1, further comprising a water-insoluble active pharmaceutical ingredient (“API”).

3. The composition of claim 2, wherein the API comprises loteprednol etabonate.

4. The composition of claim 3, wherein the viscosity is in the range of 1000 cp to about 1500 cp outside the eye.

5. The composition of claim 4, wherein said polymer is polyacryclic acid.

6. The composition of claim 5, comprising 0.2-0.5% polycarbophil, 0.3-0.6% propylene glycol, 0.6-1% glycerin, 0.3-2% loteprednol etabonate, 0.03-0.07% EDTA disodium dihydrate, 0.3-1% tyloxapol, 0.3-0.6% boric acid, 0.003-0.01% benzalkonium chloride, and water, wherein all percentages are in percent by weight of the total composition.

7. A method for preparing an ophthalmic composition, the method comprising:
(a) preparing a an aqueous solution comprising a carboxy-containing polyanionic polymer;
(b) adding a water-insoluble API into said solution to produce said composition;
wherein said composition has a viscosity in the range from about 300 cp to about 1500 cp outside the eye, a total concentration of cations of less than 50 mM, and the viscosity does not increase upon contacting surface ocular fluid.

8. The method of claim 7, wherein said API is loteprednol etabonate.

9. The method of claim 8, wherein said polymer is polyacrylic acid.

10. The method of claim 9, wherein said viscosity is in the range from about 1000 cp to about 1500 cp.
